(19) United States
(12) Patent Application Publication
(10) Pub. No.: US 2007/0284249 A1
(43) Pub. Date: Dec. 13, 2007

(54) Microchannel cleaning method

(75) Inventors: Tetsuo Ohta, Kanagawa (JP); Seiichi Takagi, Kanagawa (JP)

Correspondence Address: OLIFF & BERRIDGE, PLC, P.O. BOX 19928, ALEXANDRIA, VA, 22320, US

(73) Assignee: FUJI XEROX CO., LTD., TOKYO (JP)

(21) Appl. No.: 11/594,956

(22) Filed: Nov. 9, 2006

(30) Foreign Application Priority Data
Jun. 8, 2006 (JP) 2006-159290

(51) Int. Cl. C07K 1/26

(52) U.S. Cl. 204/451

(57) ABSTRACT

A microchannel cleaning method includes: generating bubbles by electrolyzing a fluid flowing through a microchannel; and passing the fluid containing the bubbles through the microchannel.

### BACKGROUND

1. Technical Field

The present invention relates to a microchannel cleaning method.

2. Related Art

The fine elements or devices are typified in the microreactor that is defined as the “device that is manufactured by utilizing the fine patterning and produces a reaction in the microchannel whose equivalent diameter is 500 μm or less”. In common, these fine elements or devices possess many advantages such as small-lot production of a wide variety of products, high efficiency, low environmental burden, and the like when they are applied to the technology to execute analysis, synthesis, extraction, or separation of the material, for example. Therefore, their application to various fields is expected nowadays.

In many cases the microreactor is formed of the material such as glass, plastics, metal, silicon, or the like. In particular, the glass or the plastics is often employed to watch a behavior in the inside of the microreactor. Since the glass or the plastics is jointed to the microreactor in manufacturing, such microreactor cannot be overhauled and cleaned even when foreign matters, particles, and the like in the fluid adhere to the wall surface of the microchannel to cause a blockage.

As the microchannel cleaning method in the prior art, the method of washing the adhesives away by supplying a solvent such as a water, or the like with pressure, and the method of putting a main body of the microreactor in the ultrasonic cleaner and then cleaning such microreactor while applying a pressure by a syringe, or the like are known.

### SUMMARY

(1) According to an aspect of the present invention, a microchannel cleaning method includes: generating bubbles by electrolyzing a fluid flowing through a microchannel; and passing the fluid containing the bubbles through the microchannel.

### BRIEF DESCRIPTION OF THE DRAWINGS

Exemplary embodiment of the present invention will be described in detail based on the following figures, wherein:

FIG. 1 is a schematic view showing an example of a microreactor used in a microchannel cleaning method of the present invention;

FIG. 2 is a fragmental enlarged view of an electrode and its neighborhood in the microreactor shown in FIG. 1 when a voltage is applied to the electrode;

FIG. 3 is a schematic sectional view of the electrode and its neighborhood when an example of the microreactor used in the microchannel cleaning method of the present invention is cut in a center portion of the microchannel;

FIGS. 4A and 4B are schematic projection views showing two sheets of microreactor substrates constituting another example of the microreactor used in the microchannel cleaning method of the present invention; and

FIG. 5 is a graph showing a relationship between the conductivity (μS/cm) and the voltage (V) in the electrolysis.

### DETAILED DESCRIPTION

The present invention will be explained in detail with reference to the drawings hereunder.

A microchannel cleaning method of the present invention (referred simply to as a “cleaning method of the present invention” hereinafter) includes a step of generating bubbles by electrolyzing a fluid that flows through a microchannel (referred also to as a “bubble generating step” hereinafter); and a step of passing the fluid containing the bubbles through the microchannel (referred also to as a “bubble passing step” hereinafter).

The microchannel cleaning method of the present invention is a cleaning method that generates the bubbles in the microchannel by electrolyzing the fluid that flows through the microchannel and then passes the fluid in which the bubbles are mixed through the microchannel to remove dirt, adhesives, and the like in the microchannel. Because the bubbles are generated in the microchannel in terms of the electrolysis to execute the cleaning, the device containing the microchannel can be cleaned without an overhaul with excellent detergency of the microchannel. Also, because the microchannel is cleaned repeatedly appropriately by the cleaning method of the present invention, the microchannel can be used many times for a long term.

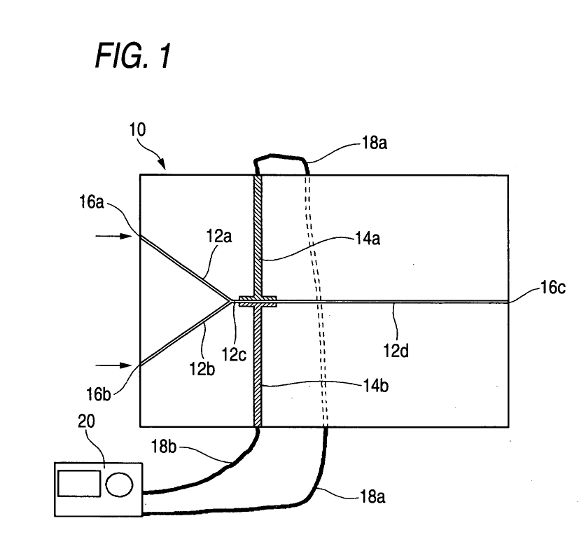

An embodiment of a microchannel cleaning method of the present invention will be explained by using a microreactor shown in FIG. 1.

FIG. 1 is a schematic view showing an example of a microreactor used in a microchannel cleaning method of the present invention.

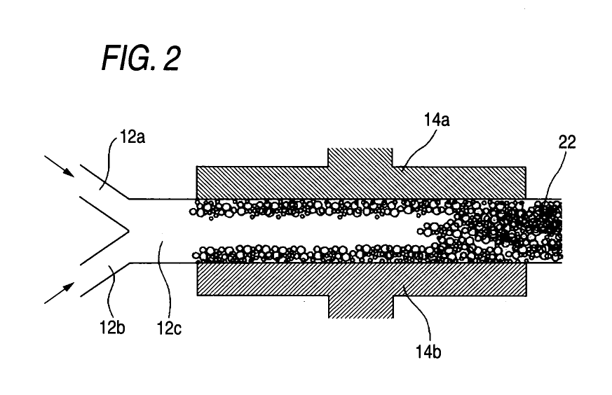

Also, FIG. 2 is a fragmental enlarged view of an electrode and its neighborhood in the microreactor shown in FIG. 1 when a voltage is applied to the electrode.

A microreactor 10 shown in FIG. 1 includes microchannels 12 (12a, 12b, 12c, and 12d), electrodes 14a and 14b provided to a part of an inner wall of the microchannel to oppose to each other in the direction that intersects orthogonally with a flow direction, and fluid introducing (recovering) portions 16 (16a, 16b, and 16c) as end portions of the microchannels. A power supply unit 20 is connected to the electrodes 14a and 14b via cords 18 and 18b respectively.

In the microreactor 10 shown in FIG. 1, components of the fluid can be electrolyzed by applying a voltage between the electrodes 14a and 14b while the fluid is supplied from the fluid introducing portion 16a and the fluid introducing portion 16b. As shown in FIG. 2, bubbles 22 are generated because the electrolysis is executed by applying a voltage between two electrodes (between the electrodes 14a and 14b). Then, the bubbles 22 as well as the fluid flow to the downstream microchannel 12d, so that the dirt, the adhesives, and the like in the microchannel 12d can be washed away by the bubbles 22 as well as the fluid and then can be eliminated from the fluid recovering portion 16c. Also, the microchannels 12a, 12b, and 12c can be cleaned by executing the similar operations as the above while the fluid is supplied conversely.

During the cleaning of the microchannel, preferably the bubbles 22 should be generated by applying a voltage between two electrodes (between the electrodes 14a and 14b) in a state that the fluid flowing through the microchannel are being fed stably like a laminar flow. Also, preferably the cleaning should be executed not continuously but intermittently at a time interval, and more preferably the cleaning should be executed every predetermined time period.

The fluid available for the microchannel cleaning method of the present invention, i.e., the fluid fed through the microchannel at a time of cleaning is not particularly restricted if at least one component of the constituents of such fluid can be electrolyzed to generate a gas. Preferably a water containing solution whose water as the constituent can be electrolyzed to generate an oxygen and/or a hydrogen should be employed. More preferably a water containing solution whose water as the constituent can be electrolyzed to generate an oxygen and a hydrogen should be employed. Most preferably a water containing solution that does not contain a halogenide ion and a heavy metal ion such as a copper ion, a silver ion, or the like should be employed.

As the fluid used in cleaning, the fluid used in purposes of reaction, mixing, etc. may be employed at a time of cleaning as it is if such fluid can generate a gas by the electrolysis, as described above. Also, the microchannel may be cleaned by using the cleaning fluid (cleaning liquid) separately. Also, as the fluid used in cleaning, the fluid containing a solid or a gas except the gas generated by the electrolysis may be employed according to the application. Also, a composition, etc. of the fluid may be selected as occasion demands.

As the fluid used in cleaning, an electrolyte solution, an acid/alkali aqueous solution, a water/alcohol dispersed solution, and the like can be listed.

Preferably an electric conductivity (referred also to as “conductivity” hereinafter. Suppose that S (siemens)/cm is used as its unit) of the fluid used in cleaning should be set to 10 μS/cm or more to execute the electrolysis easily. More preferably the electric conductivity should be set to 100 μS/cm or more. Most preferably the electric conductivity should be set to 500 to 1,000 μS/cm.

A temperature of the fluid used in cleaning is not particularly restricted. Preferably a temperature that is suited to remove a pollutant should be selected. Also, it is needless to say that a temperature at which constituent materials of the microreactor are not damaged should be selected.

Also, there is no need that the fluid that can generate a gas by the electrolysis should be selected as the fluid that is passed through the microchannel device of the present invention other than the cleaning operation. A desired solvent such as an aqueous solvent, an organic solvent, their mixture, or the like may be employed. Also, the fluid containing a solid or a gas may be employed according to the used purpose. Also, a composition, a concentration, etc. of the fluid may be selected as occasion demands.

As the material of the electrode used in the electrolysis, preferably the material that is not corroded or dissolved at a time of electrolysis should be employed. More preferably platinum or gold, which shows a weak ionization tendency, should be employed. Also, as the electrode used in the electrolysis, the electrode plated with a noble metal such as platinum, gold, or the like may be employed.

Also, in the present invention, preferably a voltage applied between two electrodes to execute the electrolysis should be set to 1.0 to 30.0 V although such voltage is different depending on type, temperature, etc. of the used fluid. More preferably the voltage should be set to 1.5 to 6.0 V. If the voltage is set in the above range, the bubbles enough to clean the microchannel can be generated and also dissolution, decomposition, etc. of the base material of the microreactor by a heat generation, or the like hardly occur. Also, further preferably the range of the voltage and the range of the electric conductivity should be combined with each other.

In the microchannel cleaning method of the present invention, the electrode structure used in the electrolysis is not particularly restricted if such structure can electrolyze at least one component of the constituent in the fluid passing through the microchannel. In order not to disturb the laminar flow passing through the microchannel, preferably the structure in which the electrodes are provided not to produce unevenness on a surface of the inner wall of the microchannel should be employed. Also, in order to execute effectively the electrolysis, preferably the electrode structure in which the electrodes are provided to oppose to the inner wall of the microchannel should be employed. Also, an electric field applying means that can apply a voltage necessary for the electrolysis is connected electrically to these electrodes. As the electric field applying means, the publicly known device, and the like can be employed. Concretely the power supply unit, the battery, and the like can be listed.

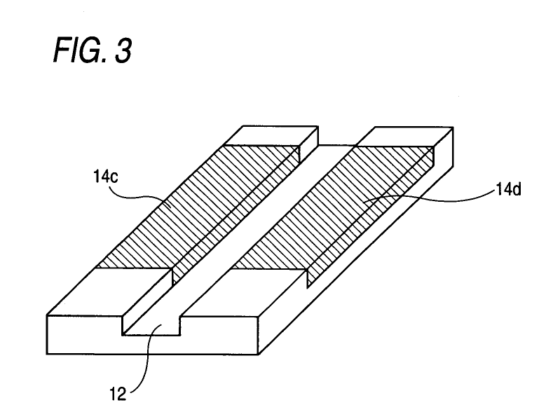

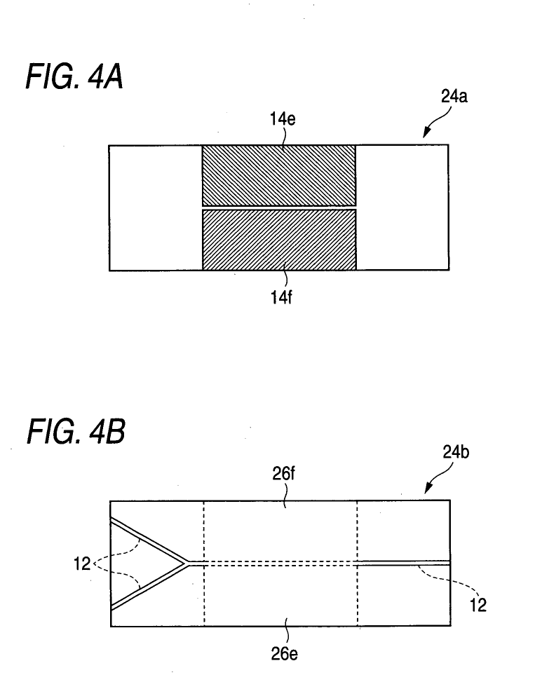

Concretely respective structures shown in FIG. 3 and FIG. 4 can be illustrated as the electrode structure, but the present invention is not limited to these structures.

FIG. 3 is a schematic sectional view of the electrode and its neighborhood when an example of the microreactor used in the microchannel cleaning method of the present invention is cut in a center portion of the microchannel.

A sectional shape of the microchannel 12 in the direction perpendicular to a flow direction is a rectangular shape around the electrode structure in the microreactor shown in FIG. 3. The electrodes 14c, 14d are provided to a part of a pair of opposing inner walls of the microchannel 12. Also, the electrodes 14c, 14d are provided like a thin film in the outward direction of the microreactor respectively, and are connected to a power supply (not shown).

FIGS. 4A and 4B are schematic projection views showing two sheets of microreactor substrates constituting another example of the microreactor used in the microchannel cleaning method of the present invention.

FIGS. 4A and 4B show microreactor substrates 24a and 24b respectively. The microreactor can be manufactured by joining two sheets of substrates.

In the microreactor substrate 24a shown in FIG. 4A, two electrodes 14e and 14f are provided on the surface in the center portion of the substrate 24a at an interval that corresponds to a diameter of the microchannel. Also, thicknesses of the electrodes 14e and 14f are set equal to depths of electrode inserting portions 26e and 26f, described later, respectively.

In the microreactor substrate 24b shown in FIG. 4B, the Y-shaped microchannel 12 is provided. Also, the electrode inserting portions 26e and 26f are provided to the center portion of the substrate 24b to have a depth same as the microchannel 12. Also, shapes of the electrode inserting portions 26e and 26f are equal to shapes of the electrodes 14e and 14f.

Two microreactor substrates 24a and 24b are fitted mutually such that the electrodes 14e and 14f are aligned with the electrode inserting portions 26e and 26f respectively, and then two substrates 24a and 24b are joined together. Thus, the microreactor that can be used in the present invention can be obtained.

A flow rate of the fluid in cleaning is not particularly restricted. This flow rate of the fluid can be adjusted appropriately in answer to an extent of the dirt or adhesives in the microchannel, a strength of the microreactor, an amount of generated bubble by the electrolysis, and the like.

An average particle diameter of the bubble generated in the microchannel by the electrolysis depends on a pressure of the fluid, a flow rate, an amount of generated bubble, and the like. However, preferably the diameter should be set to 1/50 to ½ of a channel diameter of the microchannel, and more preferably the diameter should be set to 1/20 to ¼ of the same. When the average particle diameter is set within the above range, a pollutant, a deposit, and a blocking substance can be washed away more easily.

As the method of measuring the average particle diameter of the bubble, no limitation is imposed particularly and the publicly known method can be employed. For example, the method of sensing the average particle diameter by analyzing a microscopic image by means of an image analyzing system can be listed.

As the material of the microreactor that can be used in the present invention, ceramics, glass, silicon, resin, etc. can be listed, and preferably the resin can be selected. Also, as the material of the microreactor, a conductor such as a metal, or the like may be employed as a part of the microreactor. In such case, it is important that such conductor should be isolated appropriately from the electrodes not to affect the electrolysis.

Also, preferably the glass should be employed as the material from viewpoints of transparency, workability, and the like. Also, preferably the resin should be employed from viewpoints of low cost, transparency, moldability, impact resistance, and the like.

The common glass, e.g., soda glass, quartz glass, borosilicate glass, crystal glass, or the like can be employed as the glass. Also, preferably a glass transition point of the glass should be set to 500 to 600° C.

Preferably the resin whose impact resistance, thermal resistance, chemical resistance, transparency, or the like is suitable for the aimed reaction, unit operation, or the like should be employed as the resin. Concretely, preferably polyester resin, styrene resin, acrylic resin, styrene-acrylic resin, silicon resin, epoxy resin, diene resin, phenol resin, terpene resin, coumarone resin, amide resin, amide-imide resin, butyral resin, urethane resin, ethylene-vinyl acetate resin, polydimethylsiloxane and the like can be listed. More preferably, acrylic resin such as methyl methacrylate resin, or the like, styrene resin, etc. should be employed. Also, preferably the resin having a glass transition point should be employed as the resin. Also, preferably the glass transition point should be set in a range of 90 to 150° C., and more preferably such point should be set in a range of 100 to 140° C.

The micro channel is the channel formed in a microscale. That is, a width of the channel (channel diameter) is less than 5,000 μm, and preferably the width is in a range of 10 to 1,000 μm and more preferably the width is in a range of 30 to 500 μm. Also, a depth of the channel is almost in a range of about 10 to 500 μm. In addition, preferably a length of the channel should be set in a range of 5 to 400 mm although depending on a shape of the channel to be formed, and more preferably the length should be set in a range of 10 to 200 mm.

Also, a shape of the microchannel is not particularly limited. For example, a sectional shape taken in the direction perpendicular to a flow direction may be shaped into a desired shape such as a circular shape, an elliptic shape, a polygonal shape, or the like.

A size of the microreactor can be set appropriately in response to the using purpose. Preferably the size should be set in a range of 1 to 100 cm², and more preferably the size should be set in a range of 10 to 40 cm². Also, preferably a thickness of the microreactor should be set in a range of 2 to 30 mm, and more preferably the thickness should be set in a range of 3 to 15 mm.

The microreactor that can be used in the present invention may have one microchannel or more having a bubble generating means, as occasion demands, and may have a branch of the channel, a junction portion, other microchannel, and the like. Also, the publicly known cleaning means such as the means for applying a pressure to the fluid by a syringe, a pump, or the like, the ultrasonic irradiating means, etc. may be used together as the cleaning means, in addition to the bubble generating means.

The microreactor that can be used in the present invention may have a heat radiating means or a cooling means since sometimes a heat is generated by the electrode portion when the bubbles are generated by the electrolysis. Also, the microreactor of the present invention may have a heating means for use in a temperature adjustment in the electrolysis, a temperature adjustment for the purpose of reaction, or the like except the cleaning, for example.

Also, the microreactor that can be employed in the present invention may have a portion that has a function of reaction, mixing, separation, purification, analysis, cleaning by another method, or the like according to the application, in addition to the microchannel having the bubble generating means.

For example, a fluid feeding port for feeding the fluid to the microreactor, a fluid recovery port for recovering the fluid from the microreactor, etc. may be provided to the microreactor of the present invention, if necessary.

Also, preferably a plurality of microreactors of the present invention can be used in combination according to the application. Otherwise, preferably the microreactor of the present invention can be combined with a device having a function of reaction, mixing, separation, purification, analysis, or the like, another microreactor such as a fluid feeding device, a fluid recovering device, or the like, and the like. Thus, a microchemical system can be built up preferably.

A method of manufacturing the microreactor that can be used in the present invention is not particularly limited, and the publicly known method can be employed.

As the method of forming the microchannel in the microreactor, no limitation is imposed particularly and, for example, the publicly known method can be employed. The microchannel can be manufactured by the micromachining technology, for example. As the micromachining technology, there are a method using the LIGA technology using an X-ray, a method of processing a resist portion as a structure by the photolithography method, a method of forming an opening portion in the resist by the etching, a micro electric discharge machining process, a laser beam machining process using a YAG laser, a UV laser, or the like, and a mechanical micro cutting process such as an end mill, or the like using a micro tool made of a hard material such as a diamond, and the like. These technologies may be applied solely or in combination.

### EXAMPLES

The present invention will be explained with reference to Examples hereunder. But these Examples should not be interpreted to restrict the present invention at all.

### Example 1

Following operations were executed by using the microreactor shown in FIG. 1 and FIG. 2. In this case, an acrylic resin (Kuralex S (normal type) manufactured by Nitto Resin Industry Co., Ltd) was employed as the base material of the microreactor 10, the channel diameter of the microchannels 12a and 12b was set to 250 μm, the channel diameter of the microchannels 12c and 12d was set to 500 μm, the material of the electrodes 14a and 14b was a gold, the width of the electrodes 14a and 14b were set to 10 μm.

A cleaning solution (NaNo₃ solution of composition pH=about 6, an electric conductivity 100 μS/cm or more) was fed from the microchannels 12a and 12b at a flow rate of 10 ml/h to 20 ml/h by the syringe pump. The solution was fed until a flow of the cleaning solution was stabilized, and then a voltage 4.5 V was applied between the electrodes 14a and 14b. As shown in FIG. 2, the bubbles 22 were generated from the surfaces of the electrodes 14a and 14b contacting the solution to clean the inside of the channel, and thus the dirt (not shown) in the channel and the adhesives (not shown) on the wall surface of the channel were removed.

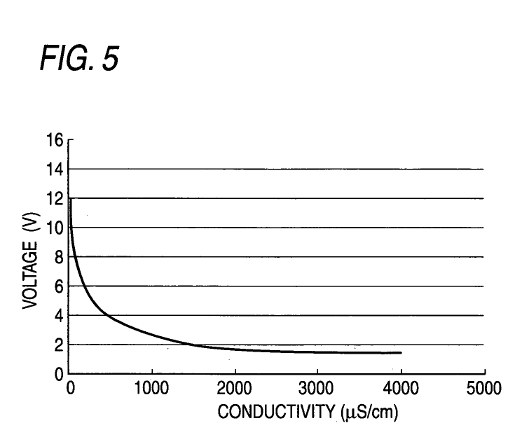

Also, it was understood that, when an electric conductivity of the cleaning solution was changed under the conditions in Example 1, the electrolysis occurred at the conductivity (μS/cm) and the voltage (V) applied in the electrolysis in an upper range of a curve in FIG. 5.

### Example 2

A photosensitive pigment synthesis was carried out by the acid pasting method while using the microreactor similar to Example 1.

A 25% concentrated ammonia solution was fed from the microchannel 12a at 10 ml/h, and a 98% concentrated sulfuric acid pigment solution was fed from the microchannel 12b at 2.0 ml/h. The ammonium sulfate generated during the synthesis was deposited on the inner wall of the channel after the fluid was fed for about 30 minute. Then, a voltage of 3.0 V was applied between the electrode 14a and the electrode 14b for about 10 second every 10 minute of fluid supply and, as shown in FIG. 2, the bubbles 22 were generated from the surfaces of the electrodes 14a and 14b contacting the solution by the electrolysis. Thus, the ammonium sulfate on the inner wall of the channel was removed by the bubbles. As a result, the fluid supply and the reaction could be conducted for 10 hours.

### Comparative Example 1

The cleaning was executed under the similar conditions to Example 2 except that the electric field was not generated and the bubble was not generated. The deposit in the channel could be removed a little, but the adhesives adhered on the wall surface of the channel could be scarcely removed.

What is claimed is:

1. A microchannel cleaning method comprising:
generating bubbles by electrolyzing a fluid flowing through a microchannel; and
passing the fluid containing the bubbles through the microchannel.

2. A microchannel cleaning method as claimed in claim 1, wherein the electrolysis is executed by applying an electric field in a direction intersecting with a flow direction of the fluid.

3. A microchannel cleaning method as claimed in claim 1, wherein the fluid is one of an electrolyte solution, one of an acid and alkali aqueous solution, and one of a water and alcohol dispersed solution.

4. A microchannel cleaning method as claimed in claim 1, wherein the fluid has an electric conductivity of 10 μS/cm to 1000 μS/cm.

5. A microchannel cleaning method as claimed in claim 1, wherein the electrolysis is executed by applying a voltage of 1.0 V to 30.0 V between two electrodes.

6. A microchannel cleaning method as claimed in claim 1, wherein the bubble has an average particle diameter of 1/50 to ½ with respect to a channel diameter of the microchannel.
